(19) United States
(12) Patent Application Publication
(10) Pub. No.: US 2026/0191561 A1
(43) Pub. Date: Jul. 9, 2026

(54) Trocar with Multi-Axis Force and Torque Sensing

(71) Applicant: ATI Industrial Automation, Inc., Apex, NC (US)

(72) Inventors: Ian Stern, Raleigh, NC (US); Joseph Lipsey, Lillington, NC (US)

(21) Appl. No.: 19/438,785

(22) Filed: Jan. 2, 2026

Related U.S. Application Data
(60) Provisional application No. 63/742,115, filed on Jan. 6, 2025.

(51) Int. Cl. A61B 17/34; A61B 34/00; G01L 1/22; G01L 5/1627; A61B 90/00

(52) U.S. Cl. CPC A61B 17/3423; A61B 34/76; G01L 1/22; G01L 5/1627; A61B 2090/064; A61B 2090/066

(57) ABSTRACT

A trocar for minimally-invasive and/or robotic surgery includes integrated multi-axis force and/or torque sensing between the outside environment and a surgical tool that will be inserted into it. The multi-axis force/torque sensing is integrated into the cannula. This allows a robotic surgical system to sense exactly how much force and/or torque the tool is applying to the skin of a patient. The multi-axis sensing is an important advance in robotic surgery, as knowing the directions of forces and torques on the trocar enables compensation for angle misalignment – which cannot be done with only single-axis force detection.

### RELATED APPLICATIONS

This application claims the benefit of U.S. Provisional Application No. 63/742115, filed January 6, 2025, the entire disclosure of which being hereby incorporated by reference herein.

### FIELD OF DISCLOSURE

The present disclosure relates generally to robotic surgical tools, and in particular to a surgical robotic trocar sensing forces and torques along multiple axes.

### BACKGROUND

The use of robotics in medical and veterinary surgery has increased dramatically. Robotics enables “minimally invasive” surgical procedures, where only one or a few small incisions are made into a patient’s body and robotic tools are inserted into the body cavity. This is a dramatic difference from conventional surgery, where an opening large enough to enable a surgeon to insert his or her hands, holding hand tools, must be cut into the patient's body. A popular form of minimally invasive surgery is laparoscopic, where a light source and camera are inserted through a small incision into the body cavity. The camera sends images of the patient’s interior to a camera monitored by a surgeon. Other surgical tools, such as scalpels, forceps, and the like, are inserted through the same or different incisions, and may be directly or remotely manipulated by the surgeon to perform surgical operations inside the body cavity. Such minimally invasive surgeries may be performed manually; with the assistance of some robotic surgical tools; or fully robotically, where the surgeon controls robotic arms that physically manipulate surgical tools within the patient’s body. In the latter case, with sufficiently high-speed and reliable data communications, the surgeon can be located remotely from the patient – in the next room or in another country.

A trocar is a medical instrument that is critical to minimally invasive surgery. A trocar may include an obturator, a cannula, and a seal. The obturator is a pointed or blunt instrument that facilitates the initial penetration into the body cavity. The cannula is a hollow tube that remains in place after the obturator is removed, providing a pathway for other surgical instruments to be inserted. The seal ensures that no air or fluids escape from the body cavity during the procedure. In laparoscopic surgery, a trocar allows a surgeon to introduce cameras and other instruments into the patient’s abdominal cavity without making large incisions, significantly reducing recovery time and the risk of complications.

It is known to integrate a force sensing system into a housing of a trocar or an inserted obturator. Such force sensing systems detects one force in one direction – along the longitudinal axis of the trocar/cannula and towards the patient. For example, the force sensing system may emit one indication (e.g., illuminates a green LED) so long as the applied force is below a predetermined threshold, and a different indication (e.g., illuminates a red LED) if the applied force exceeds the predetermined threshold. This type of force sensing system addresses the situation when a clinician may attempt to use a surgical tool (e.g., a stapler, forceps, etc.) at a position within the body cavity that is further from the incision than the length of the tool.

It is further known to attach force sensing elements, such as strain gages, to an outer sleeve of a cannula near the point of insertion into a body wall. The strain gages detect and measure force applied to the outer wall of the outer tube, by contact with the body wall, in a direction generally transverse to the longitudinal dimension of the cannula. Because the outer wall is not mechanically connected to the cannula, the strain gages are isolated from instrument-cannula interactions as instruments are manipulated within the cannula.

The Background section of this document is provided to place aspects of the present disclosure in technological and operational context, to assist those of skill in the art in understanding their scope and utility. Approaches described in the Background section could be pursued, but are not necessarily approaches that have been previously conceived or pursued. Unless explicitly identified as such, no statement herein is admitted to be prior art merely by its inclusion in the Background section.

### SUMMARY

The following presents a simplified summary of the disclosure in order to provide a basic understanding to those of skill in the art. This summary is not an extensive overview of the disclosure and is not intended to identify key/critical elements of aspects of the disclosure or to delineate the scope of the disclosure. The sole purpose of this summary is to present some concepts disclosed herein in a simplified form as a prelude to the more detailed description that is presented later.

According to one or more aspects described and claimed herein, a trocar includes integrated multi-axis force and/or torque sensing between the outside environment and a surgical tool that will be inserted into it. The multi-axis force/torque sensing is integrated into the cannula. This allows a robotic surgical system to sense exactly how much force and/or torque the tool is applying to the body wall of a patient. The multi-axis sensing is an important advance in robotic surgery, as knowing the directions of forces and torques on the trocar enables compensation for angle misalignment – which cannot be done with only single-axis force detection.

One embodiment relates to a trocar for minimally invasive, robotic, and/or robotic-assisted surgery. The trocar includes a cannula comprising a tube configured to be inserted into the body cavity of a patient. the cannula includes a multi-axis force and/or torque (F/T) sensor comprising an annular sleeve in spaced annular relationship to the tube; and at least two deformable members connecting the tube and annular sleeve. The multi-axis F/T sensor is configured to sense and resolve forces and/or torques applied between the annular sleeve and the tube along at least three axes.

Another embodiment relates to a method of performing minimally invasive surgery. An obturator of a trocar is used to insert a cannula into a patient’s body cavity. The cannula includes a tube configured to be inserted into the body cavity. The cannula also includes a multi-axis force and/or torque (F/T) sensor. The multi-axis F/T sensor includes an annular sleeve in spaced annular relationship to the tube; and at least two deformable members connecting the tube and annular sleeve. The multi-axis F/T sensor is configured to sense and resolve forces and/or torques applied between the annular sleeve and the tube along at least three axes. The method further includes performing a surgical procedure through the cannula while monitoring forces and/or torques applied by a surgical tool to the cannula.

### BRIEF DESCRIPTION OF THE DRAWINGS

The present disclosure will now be described more fully hereinafter with reference to the accompanying drawings, in which aspects of the disclosure are shown. However, this disclosure should not be construed as limited to the aspects set forth herein. Rather, these aspects are provided so that this disclosure will be thorough and complete, and will fully convey the scope of the disclosure to those skilled in the art. Like numbers refer to like elements throughout.

FIG. 1 is a plan view of a prior art robotic force/torque sensor.

FIG. 2 is an enlarged and exaggerated view of a deformable beam of the F/T sensor of FIG. 1 under an applied force.

FIG. 3 is a perspective view of a cannula of a trocar for robotic surgery according to a first embodiment.

FIG. 4 is a top plan view of the cannula of FIG. 3.

FIG. 5 is an isometric cross section view of the cannula of FIGS. 3 and 4.

FIG. 6 is a side sectional view of a cannula according to a second embodiment.

FIG. 7 is an isometric sectional view of the cannula of FIG. 6.

FIG. 8 is a side view of the cannula of FIGS. 6 and 7.

FIG. 9 is a top plan view of the cannula of FIGS. 3-5, with transducers and a controller.

FIG. 10 is a flow diagram of a method of performing minimally invasive surgery.

### DETAILED DESCRIPTION

For simplicity and illustrative purposes, the present disclosure is described by referring mainly to an exemplary aspect thereof. In the following description, numerous specific details are set forth in order to provide a thorough understanding of the present disclosure. However, it will be readily apparent to one of ordinary skill in the art that the present disclosure may be practiced without limitation to these specific details. In this description, well known methods and structures have not been described in detail so as not to unnecessarily obscure the present disclosure.

In robotic surgery, there is a need for far more, and more detailed, force and torque sensing by a trocar than the single-axis, single-direction force sensor known in the prior art. For example, with real-time feedback of forces and torques along multiple axes, surgeons can adjust their technique to avoid exerting excessive pressure in any of multiple directions, which can cause tissue damage. Such multi-axis force and/or torque sensing and feedback can significantly enhance the safety and effectiveness of minimally invasive surgeries. One of the primary benefits is a reduction in the risk of injury to internal organs and blood vessels, both during the insertion process and also during the surgical operation.

Additionally, multi-axis force/torque sensing improves the precision of surgical procedures. With accurate force measurements, surgeons can better navigate through different tissue layers, ensuring that the trocar is placed correctly and securely. This precision is particularly important in complex surgeries, where the margin for error is minimal. Enhanced control over the insertion process can lead to fewer complications and a smoother overall procedure.

Moreover, multi-axis force/torque sensing technology in a trocar facilitates the development of new types of robotic-assisted or fully robotic surgeries. By incorporating force/torque feedback into robotic systems, it becomes possible to automate certain aspects of the surgical procedure, potentially increasing the consistency and reliability of operations such as trocar insertion. This advancement also aids in training new surgeons, providing them with valuable feedback to refine their techniques and improve their skills. A multi-axis force/torque sensor also provides information that can be incorporated into the surgeon’s controls, such as through a haptic feedback system that provides force/torque feedback to the surgeon. A trocar having multi-axis force/torque sensing is beneficial to all minimally invasive surgery – including manual, robotic-assisted, and fully robotic operations.

Multi-axis force and/or torque (F/T) sensors are known in the art. For example, U.S. Patent Nos. 10,422,707; 10,067,019; 11,892,364; 11,747,224; 11,491,663; 11,085,838; and published U.S. Patent Application No. 2023/0049155, all assigned to the assignee of the present disclosure and incorporated herein by reference in their entireties, describe numerous aspects of such F/T resolution.

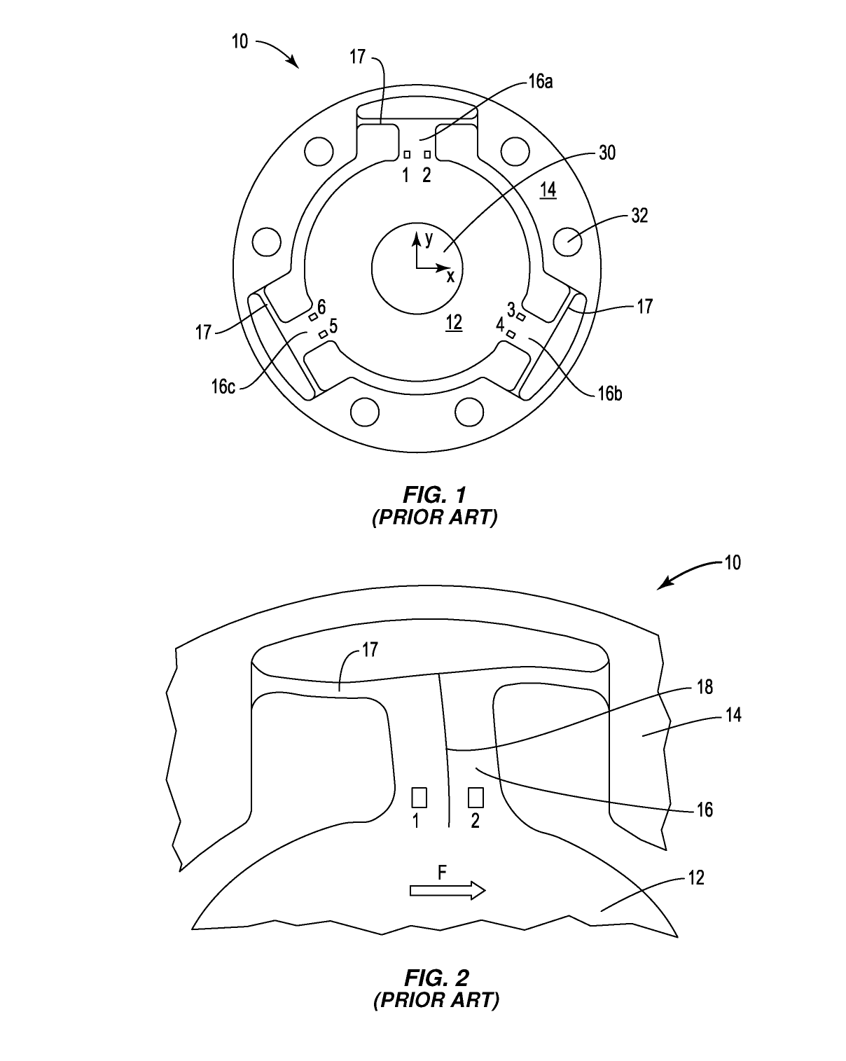

FIG. 1, which is a reproduction of FIG. 1 of the ‘707 patent incorporated above, shows a plan view of one embodiment of a force/torque sensor 10. A rigid hub 12 is connected to a rigid annular ring 14 by three deformable beams 16a, 16b, 16c. In the embodiment depicted, each beam 16 connects directly to the hub 12, and connects to the annular ring 14 by flexures 17, which aid in the deformation of the beams 16 under mechanical loading.

Affixed to the upper surface of each deformable beam 16 are strain gages 1-6. FIG. 1 also depicts two axes of a 3-dimensional reference Cartesian coordinate system (z-direction extending out of the figure), to which resolved forces and torques are referenced.

FIG. 2, which is a reproduction of FIG. 2 of the same ‘707 patent, is an enlarged view of one beam 16a, showing an exaggerated deformation due to a force F applied to the hub 12, relative to the annular ring 14. This force deforms the beam 16a slightly to the left (the figure is not to scale). A compressive force is induced on the left side surface of deformable beam 16a, and a tensile force is induced on the right side surface. Transducers, such as strain gages, may be mounted on these surfaces, where they generate strong signals, of opposite polarity, from which the deformation, and hence the applied force F, may be ascertained. In embodiments of the ‘707 patent, the strain gages 1-6 are mounted only on an upper surface of the deformable beams 16, spaced apart from a neutral axis 18. The ‘707 patent, as well as other of the patents incorporated above, describe the signal processing by which multi-axis forces and/or torques between the hub 12 and annular ring 14 are resolved and quantified.

In the field of minimally invasive surgery, there is a need to accurately sense and measure forces and torques applied by a patient’s body wall to the cannula of a trocar. As a surgeon manipulates instruments inserted into a patient through a cannula, he or she may create a lever action, with the patient’s body acting as the fulcrum of the lever, at the point of incision in the body wall. Such forces apply stress to the body wall tissue, which can cause tissue trauma. There is a need to monitor these forces, in part so that damage to body wall tissue can be minimized.

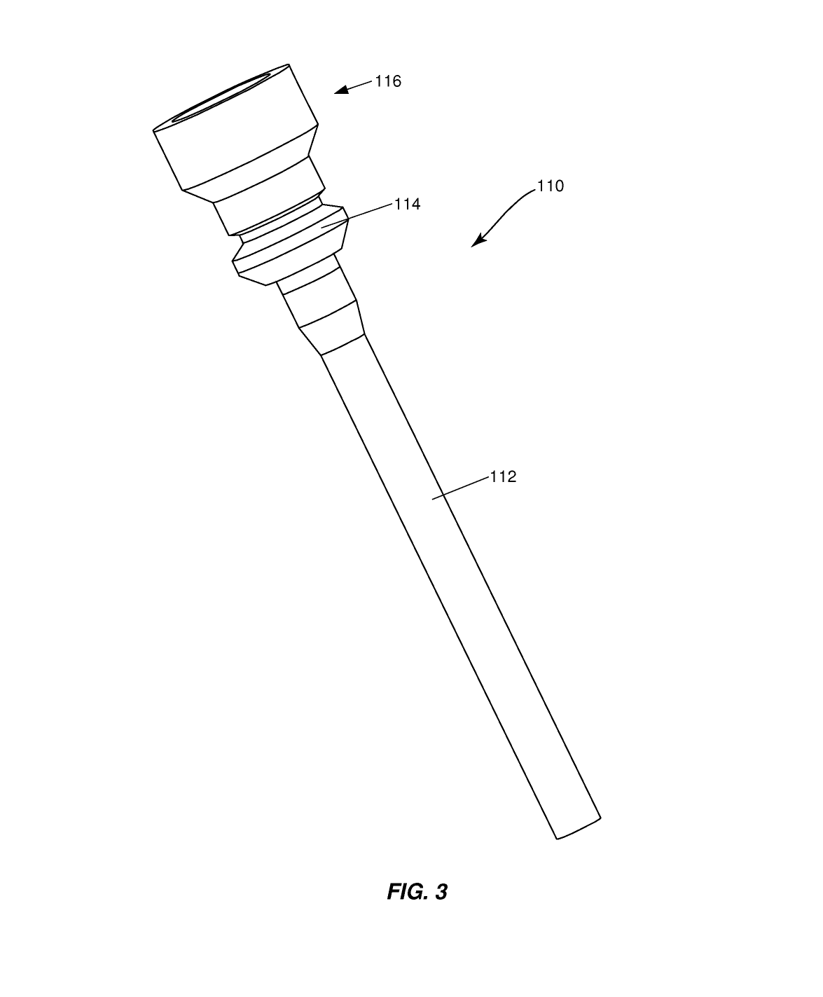

FIG. 3 depicts a perspective view of a cannula 110 for a trocar for minimally invasive surgery. The cannula 100 includes an elongated tube 112, which during minimally invasive surgery is inserted into a patient’s body cavity through a small incision. Surgical tools are inserted into the body cavity through the tube 112 of the cannula 110. An inner seal 114 is positioned within the patient’s body, just below the skin. The inner seal 114 prevents gas or fluid from escaping through the incision during surgical operations. A multi-axis F/T sensor 116 is disposed on the end of the cannula 110, opposite the inner seal 114.

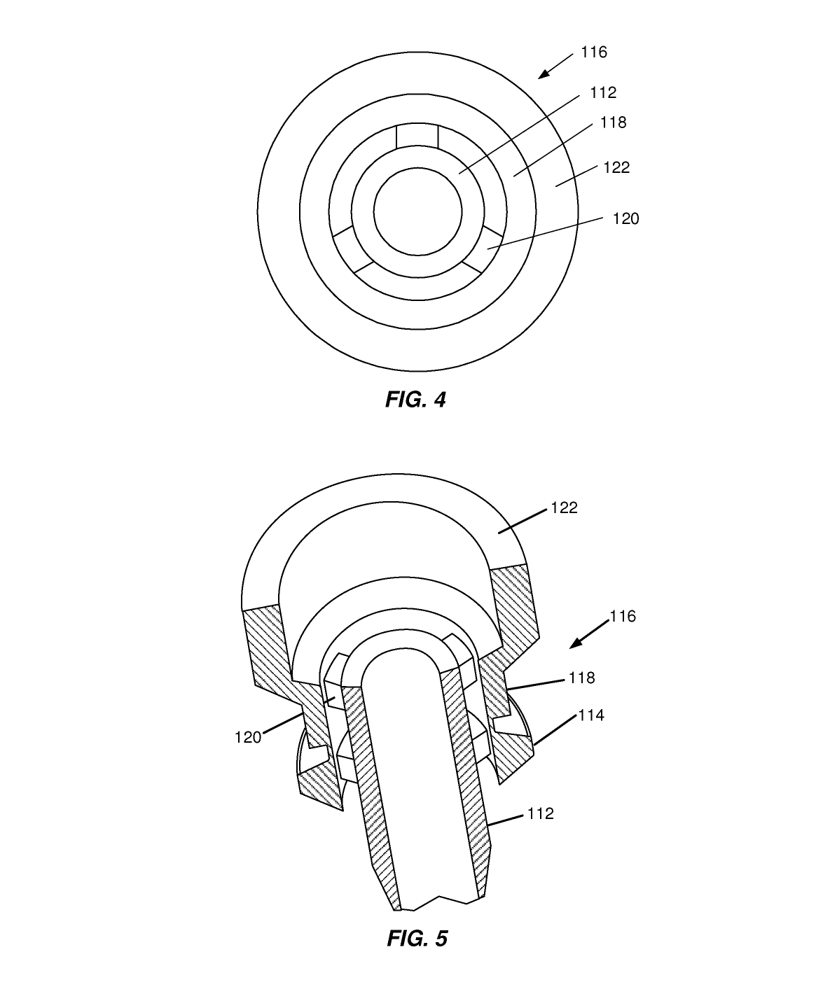

FIGS. 4 and 5 show details of the multi-axis F/T sensor 116. An annular sleeve 118 extends around the tube 112, at least partially along its length. The tube 112 and annular sleeve 118 are relatively rigid, and are connected at a plurality of radial positions – in some embodiments, resembling the spokes on a wheel – by instrumented deformable members 120. The instrumented deformable members 120 deform slightly in response to forces and/or torques between the tube 112 and annular sleeve 118. Transducers (not shown) affixed to one or more surfaces of a plurality of deformable members 120 sense tensile and/or compressive forces induced on surfaces of the deformable members 120 by the applied forces and/or torques, and generate outputs, such as electrical signals. These are transmitted, such as by wires (not shown) to a controller (not shown), which resolves the sensed tensile and compressive forces into forces and/or torques along a plurality of axes. In one embodiment, an extended annular member 122 provides space for an inner seal or other components to be fitted onto the cannula 110.

As best seen in FIG. 5, in one embodiment the instrumented deformable members 120 are formed in two sets of three, which sets are laterally spaced apart from each other along the length of the tube 112. In various embodiments, any number of instrumented deformable members 120 in each set may be radially spaced around the tube 112, and there may be more than two sets of instrumented deformable members 120 spaced along the tube 112.

Regardless of the specific configuration of instrumented deformable members 120, the multi-axis F/T sensor 116 is configured to sense and resolve forces and/or torques between the tube 112 and the annular sleeve 118. In a surgical operation, the annular sleeve 118 is held in place on the patient’s body, and surgical instrument are inserted through the tube 112. Forces and/or torques between the tube 112 and annular sleeve 118, such as those induced by a surgeon and/or robot manipulating a surgical tool disposed in the tube 112, are sensed, resolved, and reported (and/or fed back to a haptic feedback system) by the multi-axis F/T sensor 116.

In general, enough transducers are attached to the deformable members 120 to resolve between three and six axes of forces and torques. At least one instrumented deformable member 120 is required per resolved axis; however, each instrumented deformable member 120 may have multiple transducers affixed thereto.

In the embodiment shown in FIGS. 4 and 5, there are two sets of three instrumented deformable members 120 connecting the tube 112 and annular sleeve 118. At least two instrumented deformable members 120 are required. In the depicted embodiment there are six total deformable members 120 for strength purposes; however, a typical embodiment may include only three or four. The instrumented deformable members 120 may be more complicated than the simple beams depicted. For example, they may be L-shaped or T-shaped, and/or may include flexures as shown in FIGS. 1 and 2 and described in the ‘707 patent. The instrumented deformable members 120 may also be parallel or tangential to the tube 112 instead of radial, as shown.

In the embodiment depicted in FIG. 5, the annular sleeve 118 extends along the length of the tube 112 only for a distance sufficient to encompass all (e.g., both sets of) instrumented deformable members 120. In another embodiment (not shown), the annular sleeve 118 extends along the entire length of the tube 112. This isolates the tube 112 from forces that are seen by external factors that are unrelated to force applied to the tube 112. However, it requires a larger incision, as the outer diameter of the cannula 110 is now larger.

As described in the above incorporated patents, the transducers affixed to the deformable members 120 may comprise strain gages, such as silicon strain gages. However, the invention is not limited to strain gages. In other embodiments, the transducers may comprise capacitive, fiber bragg grating, Surface Acoustic Wave (SAW), or piezoelectric sensors, or combinations thereof.

The cannula 110 may be formed as a single-piece system, or alternatively may comprise two or more modular pieces that are assembled into an operative cannula 110.

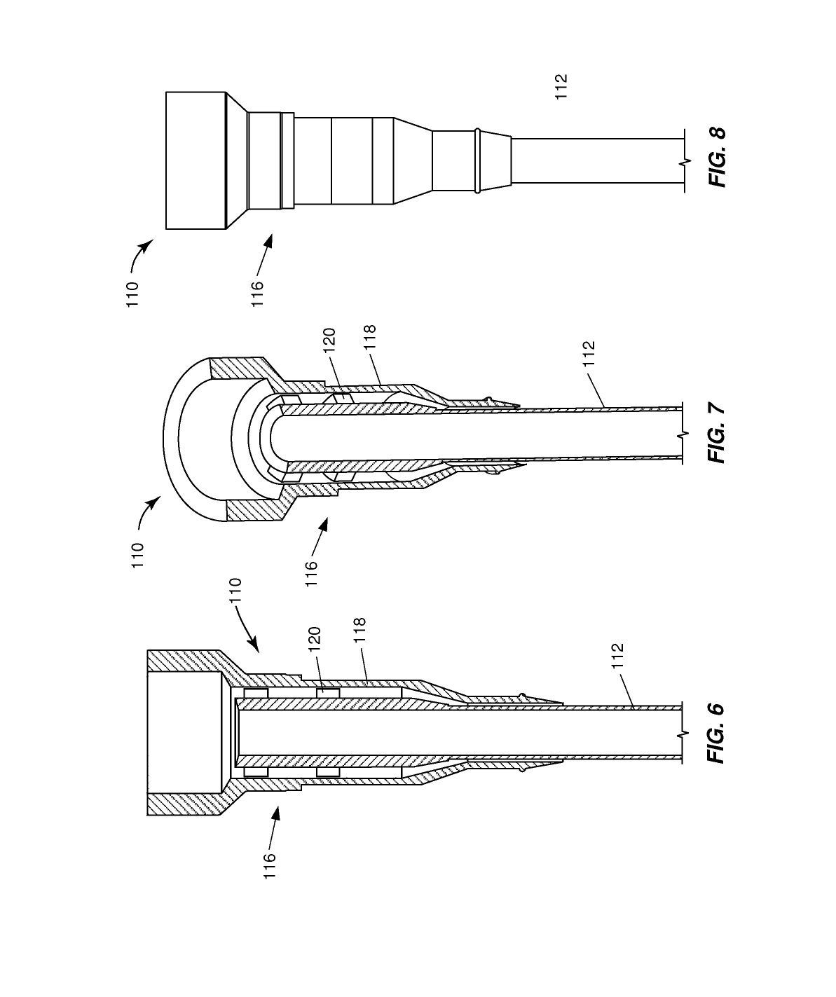

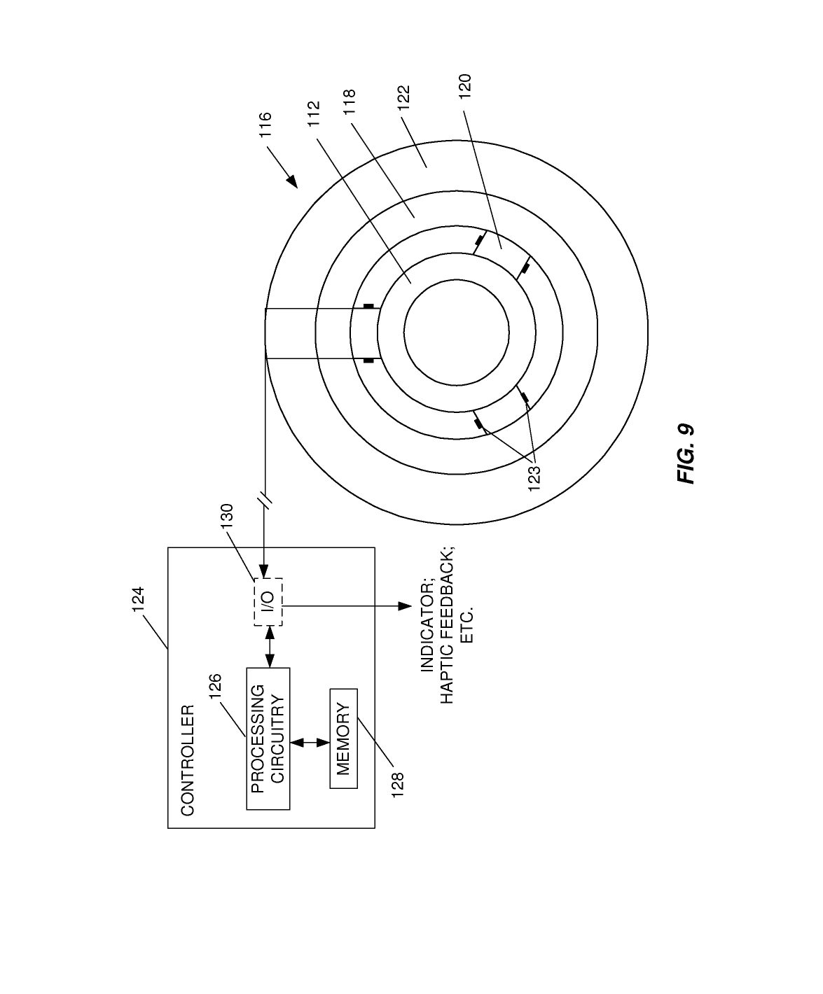

FIGS. 6, 7, and 8 show side sectional, isometric sectional, and side views, respectively, of a cannula 110 for a minimally invasive surgical trocar according to another embodiment. In this embodiment, the tube 112 and the annular sleeve 118 are closer together, and the tube 112 has a thicker wall at the distal end where it connects to the annular sleeve 118. In general, it is advantageous to minimize the overall outer diameter of the cannula 110. The multi-axis F/T sensor 116 operates as described above.

FIG. 9 depicts the cannula 110 of FIG. 3, showing transducers 123, in this embodiment strain gages, connected to the side walls of deformable members 120. In other embodiments, other transducers 123 may be used, and/or they may be attached to the deformable members 120 in different locations. In one embodiment, one or more transducers 123 may be attached to the tube 112 or annular sleeve 118 proximate to a deformable member 120.

The transducers 123 are operatively connected to a controller 124 (only one pair shown connected in FIG. 9). The controller 124 includes processing circuitry 126 operatively connected to memory 128, and optionally (as indicated by dashed lines) input/output (I/O) circuitry 130. The controller 124 may be integrated into the cannula 110 or trocar, or may be part of a laparoscopic or robotic surgical system.

The processing circuitry 126 is configured to receive electrical signals from transducers 123, and to resolve the signals into forces and/or torques applied between the annular sleeve 118 and the tube 112 along at least three axes. Algorithms for resolving transducer outputs into forces and torques are known in the art, and are disclosed, for example, in the above incorporated patents. These algorithms are embodied in machine-readable code stored in memory 128, which is accessed by the processing circuitry 126.

Optional I/O circuitry may pre-process the transducer 123 output signals prior to their processing by the processing circuitry 126, such as by amplifying, low-pass filtering to reduce noise, and performing analog to digital conversion. The I/O circuity may also post-process the resolved forces and torques, such as by comparing them to predetermined thresholds to emit an alarm if excessive force or torque is detected, by transforming the F/T to a format required by haptic feedback circuitry in a surgical controller, or for other uses. Alternatively, the processing circuitry 126 may perform all formatting of the output data.

The processing circuitry 126 may comprise any sequential state machine operative to execute machine instructions stored as machine-readable computer programs in memory 128, such as one or more hardware-implemented state machines (e.g., in discrete logic, FPGA, ASIC, etc.), programmable logic together with appropriate firmware; one or more stored-program, general-purpose processors, such as a microprocessor or Digital Signal Processor (DSP), together with appropriate software; or any combination of the above.

The memory 128 may comprise any non-transitory machine-readable media known in the art or that may be developed, including but not limited to magnetic media (e.g., floppy disc, hard disc drive, etc.), optical media (e.g., CD-ROM, DVD-ROM, etc.), solid state media (e.g., SRAM, DRAM, DDRAM, ROM, PROM, EPROM, Flash memory, solid state disc, etc.), or the like.

The optional I/O circuitry 126 may comprise receiver circuitry configured to receive, amplify, and digitize electrical signals from transducers 123. The I/O circuitry may additionally include transmitter or interface circuitry configured to output useful force/torque data to other systems or components, such as an excessive force indicator (e.g., flashing light, audible alarm, or the like), as data for a haptic feedback system, or the like.

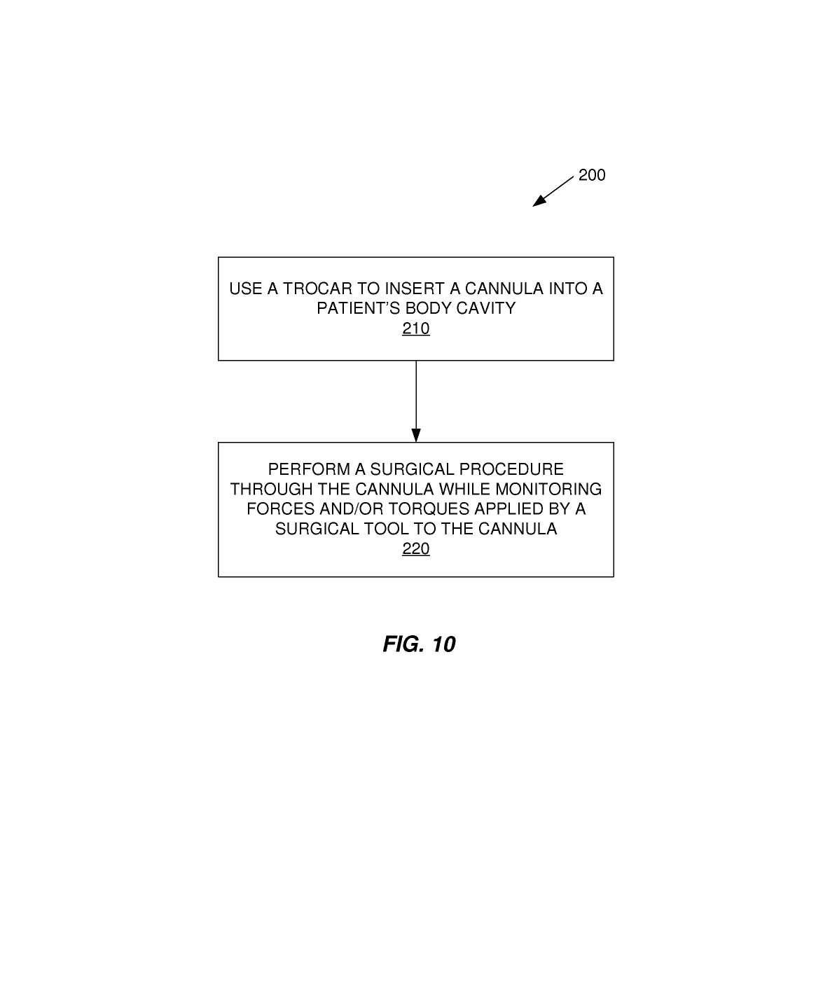

FIG. 10 depicts the steps in a method 200 of performing minimally invasive surgery. A trocar is used to insert a cannula 110 into a patient’s body cavity (block 210). The cannula 110 includes a tube 112 configured to be inserted into the body cavity, and also includes a multi-axis force and/or torque (F/T) sensor 116. The multi-axis F/T sensor includes an annular sleeve 118 in spaced annular relationship to the tube 112, and at least two instrumented, deformable members 120 connecting the tube 112 and annular sleeve 118. The multi-axis F/T sensor 116 is configured to sense and resolve forces and/or torques applied between the annular sleeve 118 and the tube 112 along at least three axes. A surgical procedure is performed through the cannula 110 while monitoring forces and/or torques applied by a surgical tool to the cannula 110 (block 220).

Embodiments of the present disclosure present significant advantages over the prior art, and may achieve one or more of the following technical effects. Force-only sensing does not have the ability to differentiate directional forces and torques. Robotic surgery controls built around multi-axis sensing are much more accurate, and have greater fault recognition. Also, a torque that is in the same axis as the tube cannot be sensed by force-only sensing, since no forces are applied to the tool, only a pure torque. Furthermore, a surgical tool that is in the tube will have multiple contact points if the tool is not in the same axis as the tube . These cause multiple points of contact may create equal and opposite forces, which may cancel out in a single-axis force detector, thus providing no F/T feedback for the control system.

Generally, all terms used herein are to be interpreted according to their ordinary meaning in the relevant technical field, unless a different meaning is clearly given and/or is implied from the context in which it is used. All references to a/an/the element, apparatus, component, means, step, etc. are to be interpreted openly as referring to at least one instance of the element, apparatus, component, means, step, etc., unless explicitly stated otherwise. Any feature of any of the aspects disclosed herein may be applied to any other aspect, wherever appropriate. Likewise, any advantage of any of the aspects may apply to any other aspects, and vice versa. Other objectives, features, and advantages of the enclosed aspects will be apparent from the description.

As used herein, the term “configured to” means set up, organized, adapted, or arranged to operate in a particular way; the term is synonymous with “designed to,” or in the case of processing circuitry and/or software, “programmed to.” As used herein, a “surgical tool” is any device that may be inserted into a patient’s body cavity during minimally-invasive surgery, whether it performs a specific surgical function or supports other surgical tools. For example, a laparoscopic camera is a surgical “tool,” even though it does not directly interact with the patient’s body. As used herein, and “instrumented” deformable beam is a beam with one or more sensors attached and configured to detect and measure deformation of the beam under applied force and/or torque loads. As used herein, the coordinating conjunction “or” has the meaning of the Boolean logical operator OR – for example, “A or B” is true if A is true, B is true, or both A and B are true; it is false only if both A and B are false. Hence, the term “or” subsumes the common phrase “and/or.”

The present disclosure may, of course, be carried out in other ways than those specifically set forth herein without departing from essential characteristics of the disclosure. The present aspects are to be considered in all respects as illustrative and not restrictive, and all changes coming within the meaning and equivalency range of the appended claims are intended to be embraced therein.

1. A trocar for minimally invasive, robotic, or robotic-assisted surgery, comprising:
a cannula comprising a tube configured to be inserted into the body cavity of a patient; and
a multi-axis force or torque (F/T) sensor comprising
an annular sleeve in spaced annular relationship to the tube; and
at least two instrumented, deformable members connecting the tube and annular sleeve;
wherein the multi-axis F/T sensor is configured to sense and resolve forces or torques applied between the annular sleeve and the tube along at least three axes.

2. The trocar of claim 1, wherein the multi-axis F/T sensor comprises two or more sets of deformable members, each set comprising at least two deformable members connecting the tube and annular sleeve and radially spaced around a longitudinal axis of the tube, and wherein each set is spaced apart along the tube in the direction of the longitudinal axis of the tube.

3. The trocar of claim 2, wherein each set of deformable members comprises three deformable members evenly spaced radially around the tube.

4. The trocar of claim 1, further comprising:
transducers affixed to one or more of the tube, deformable members, and annular sleeve, the transducers configured to sense tensile and compressive forces and output signals responsively; and
a controller configured to receive transducer output and resolve forces or torques applied between the annular sleeve and the tube along at least three axes.

5. The trocar of claim 4, wherein the controller comprises:
processing circuitry configured to resolve forces or torques applied between the annular sleeve and the tube along at least three axes, in response to transducer outputs; and
memory operatively connected to the processing circuitry, and configured to store machine-readable instructions which, when executed on the processing circuitry, cause it to resolve the forces or torques from the transducer signals.

6. The trocar of claim 4, wherein the transducers comprise strain gages.

7. The trocar of claim 4, wherein the transducers comprise one or more of capacitive sensors, fiber bragg grating sensors, Surface Acoustic Wave (SAW) sensors, and piezoelectric sensors.

8. A method of performing minimally invasive surgery, comprising:
inserting a cannula of a trocar into a patient’s body cavity, wherein the cannula comprises a tube configured to be inserted into the body cavity, the cannula including a multi-axis force or torque (F/T) sensor comprising
an annular sleeve in spaced annular relationship to the tube and
at least two instrumented, deformable members connecting the tube and annular sleeve,
wherein the multi-axis F/T sensor is configured to sense and resolve forces or torques applied between the annular sleeve and the tube along at least three axes; and
performing a surgical procedure through the cannula while monitoring forces or torques applied by a surgical tool to the cannula.

9. The method of claim 8 wherein monitoring forces or torques applied by a surgical tool to the cannula comprises receiving a visual or audible alarm if an applied force or torque exceeds a predetermined threshold.

10. The method of claim 8 wherein monitoring forces or torques applied by a surgical tool to the cannula comprises receiving haptic feedback to a surgical tool based on the sensed and resolved forces or torques.

11. The method of claim 8 wherein inserting a cannula of a trocar into a patient’s body cavity comprises using an obturator of the trocar to insert the cannula into the patient’s body cavity.
